(19) United States
(12) Patent Application Publication
(10) Pub. No.: US 2017/0303885 A1
(43) Pub. Date: Oct. 26, 2017

(54) RADIOGRAPHIC CAPTURING APPARATUS AND RADIOGRAPHIC CAPTURING SYSTEM

(71) Applicant: KONICA MINOLTA, INC., Tokyo (JP)

(72) Inventor: Tetsu HOSOKI, Tokyo (JP)

(73) Assignee: KONICA MINOLTA, INC., Tokyo (JP)

(21) Appl. No.: 15/469,257

(22) Filed: Mar. 24, 2017

(30) Foreign Application Priority Data
Apr. 26, 2016 (JP) 2016-087680

(51) Int. Cl. A61B 6/00

(52) U.S. Cl. CPC A61B 6/542; A61B 6/4064; A61B 6/4405

(57) ABSTRACT

A portable radiographic capturing apparatus includes: a detecting unit provided with a two-dimensional array of radiation detecting elements accumulating charges in proportion to a dose of radiation; and a control unit which controls accumulation of the charges by the radiation detecting elements and reading of the accumulated charges therefrom, the charges being in proportion to the dose of radiation emitted as pulsed radiation from a radiation source and transmitted through a subject, to generate plural frame images of the subject, wherein the control unit obtains waveform information on the emitted radiation by causing at least some of the radiation detecting elements to accumulate the charges in proportion to the radiation preliminarily emitted from the radiation source for adjustment and by reading the accumulated charges, and adjusts a control condition for generation of the frame images of the subject on the basis of the obtained waveform information.

### CROSS-REFERENCE TO RELATED APPLICATIONS

This application is based upon and claims the benefit of priority from the prior Japanese Patent Application No. 2016-087680 filed on Apr. 26, 2016, the entire contents of which are incorporated herein by reference.

### BACKGROUND OF THE INVENTION

### Field of the Invention

The present invention relates to a radiographic capturing apparatus and a radiographic capturing system.

### Description of Related Art

A radiographic capturing system has been recently known that includes a portable radiographic capturing apparatus (flat panel detector (FPD)) provided with a two-dimensional array of radiation detecting elements in which charges are accumulated in proportion to radiation emitted from a radiation source and transmitted through a subject. The radiographic capturing apparatus reads out the charges accumulated in the radiation detecting elements to generate image data. Such a radiographic capturing system requires synchronization between a period (referred to as a radiation irradiation period) during which a radiation source emits radiation, and a charge accumulation period (referred to as an accumulation period) during which the charges are accumulated in the radiographic capturing apparatus, such that the radiation source emits radiation during the accumulation period of the radiographic capturing apparatus.

Unfortunately, if the radiographic capturing apparatus wirelessly communicates with the radiation control device controlling the radiation source, such a communication involves a problem with real-time property. For this reason, the radiographic capturing apparatus cannot sometimes be synchronized with the radiation control device if synchronous communication is performed therebetween every radiation irradiation during video capture involving irradiation (pulse irradiation) of pulsed radiation at a predetermined time interval to obtain multiple frame images.

PTL 1 (Japanese Patent Application Laid-Open Publication No. 2010-81960) discloses a technique of generating image data indicating a radiographic image with a console as a radiation control device which instructs image capturing and an electronic cassette which incorporates an FPD. The console includes a clocking unit for counting time, and the electronic cassette includes a clocking unit for counting time in synchronization with the clocking unit of the console. A radiation source emits radiation for a predetermined period when an exposure starting time comes, the time being predetermined at the console. The charges accumulated in the FPD of the electronic cassette are read after elapse of a predetermined time from the exposure starting time, and the image data indicating a radiographic image is generated from the read charges.

Unfortunately, PTL 1 fails to take into consideration output characteristics of the radiation which is output from the radiation source. Thus, if radiation irradiation is not completed within the accumulation period due to fluctuation in the waveform of the radiation which is output from the radiation source, there is a problem that the radiation irradiation outside of the accumulation period causes degradation of the image. If a waveform cycle of the radiation which is output from the radiation source is short with respect to the accumulation period, there is a problem that a capturing time becomes ineffectively long.

### SUMMARY OF THE INVENTION

An object of the present invention is to prevent drawbacks caused by a time difference between the radiation irradiation period of the radiation source and the accumulation period of the radiographic capturing apparatus.

To achieve the above object, a portable radiographic capturing apparatus in which one aspect of the present invention is reflected includes: a detecting unit provided with a two-dimensional array of radiation detecting elements each of which accumulates one or more charges in proportion to a dose of radiation; and a control unit which controls accumulation of the charges by the radiation detecting elements and reading of the accumulated charges from the radiation detecting elements, the charges being in proportion to the dose of radiation emitted as pulsed radiation from a radiation source and transmitted through a subject, to generate a plurality of frame images of the subject, wherein the control unit obtains waveform information on the radiation emitted from the radiation source by causing at least some of the radiation detecting elements to accumulate the charges in proportion to the radiation preliminarily emitted from the radiation source for adjustment and by reading the accumulated charges, and adjusts a control condition for generation of the frame images of the subject on the basis of the obtained waveform information.

To achieve the above object, a radiographic capturing system in which one aspect of the present invention is reflected includes: a radiation source capable of emitting pulsed radiation; and the above radiographic capturing apparatus.

### BRIEF DESCRIPTION OF THE DRAWINGS

The present invention will become more fully understood from the detailed description given hereinbelow and the appended drawings, and thus are not intended as a definition of the limits of the present invention, and wherein:

FIG. 1 illustrates an overall configuration of a radiographic capturing system;

FIG. 2 is a block diagram illustrating a functional configuration of a radiation control device;

FIG. 3 is block diagram illustrating a functional configuration of an FPD cassette;

FIG. 4 illustrates examples of an operation of the FPD cassette, an amount of read charges, and a radiation tube voltage, during calibration and capturing in a first embodiment;

FIG. 5 illustrates examples of an operation of the FPD cassette, an amount of read charges, and a radiation tube voltage, during calibration and capturing in a second embodiment; and

FIG. 6 illustrates example image correction factors.

### PREFERRED EMBODIMENT OF THE PRESENT INVENTION

### First Embodiment

### (Configuration of Radiographic Capturing System 100)

The configuration according to the first embodiment of the present invention will now be described.

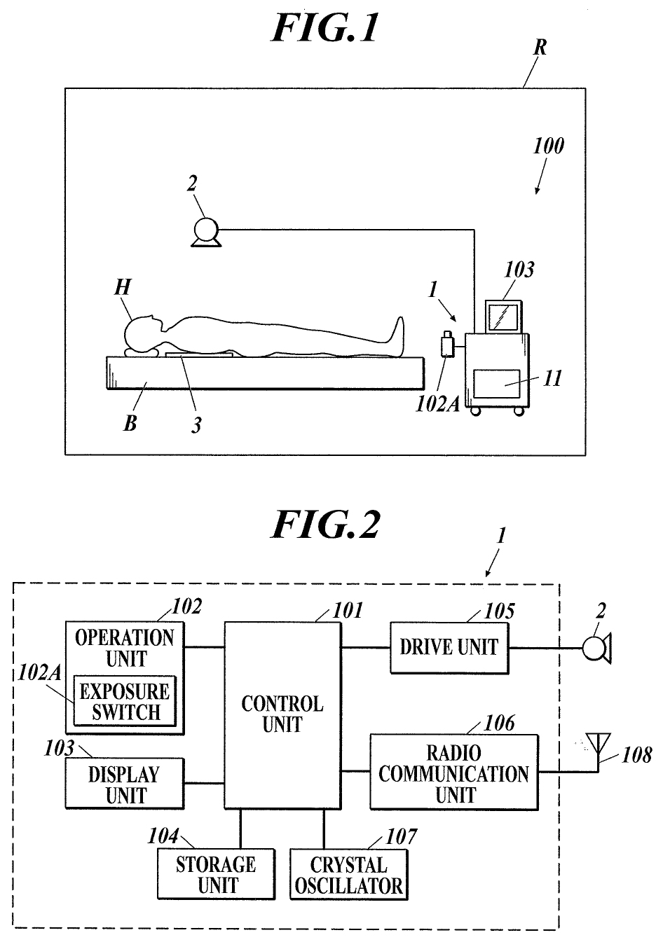

FIG. 1 illustrates the overall configuration of a radiographic capturing system 100 according to this embodiment.

The radiographic capturing system 100 is a system for round which executes radiographic capturing of patients having difficulties in moving about, for example. The radiographic capturing system 100 includes a radiation control device 1, a radiation source 2, and a flat panel detector (FPD) cassette 3. The radiation control device 1 is constructed as a mobile medical cart having wheels.

With reference to FIG. 1, the radiographic capturing system 100 is carried into a room R, such as an operating room, an intensive care unit, or a patient room. The FPD cassette 3 is placed between a bed B and a subject H laying on the bed B, or inserted into a slot (not shown) in the bed B on a face opposite to a face on which the subject H is laying, for example. In the state, the radiographic capturing system 100 causes the radiation source 2 to emit radiation to carry out video capturing of the subject H. In this embodiment, “video capturing” refers to capturing of a moving image of the subject H through repeated irradiation (pulse irradiation) of radiation such as X-rays to the subject H at a predetermined time interval, in response to a single capturing operation (an operation of an exposure switch 102A). The series of images obtained through video capturing is collectively referred to as a “moving image.” Each of the images constituting the moving image is referred to as a “frame image.”

The components of the radiographic capturing system 100 will now be described.

The radiation control device 1 controls the radiation source 2 under input radiation irradiation conditions. With reference to FIG. 2, the radiation control device 1 includes a control unit 101, an operation unit 102, a display unit 103, a storage unit 104, a drive unit 105, a radio communication unit 106, and a crystal oscillator 107.

The control unit 101 includes a central processing unit (CPU) and a random access memory (RAM). The CPU of the control unit 101 reads the system program and various processing programs stored in the storage unit 104 in response to an operation of the operation unit 102, expands these programs to the RAM, and controls the operation of the components of the radiation control device 1 in accordance with the expanded programs.

The operation unit 102 includes a touch panel including a grid of transparent electrodes and covering the surface of the display unit 103. The operation unit 102 detects the position of a finger of a user or a stylus on the touch panel and outputs the positional information as operation information to the control unit 101.

The operation unit 102 includes an exposure switch 102A that is operated by the operator to instruct to execute the radiation irradiation. The exposure switch 102A is a two-stage switch.

The display unit 103 is composed of a monitor, such as a liquid crystal display (LCD) or a cathode ray tube (CRT), and displays images in accordance with display signals from the control unit 101.

The storage unit 104 is composed of a non-volatile semiconductor memory, hard disk, or the like. The storage unit 104 stores various programs executed by the control unit 101, parameters required for the execution of the programs, and data such as processed results, etc.

The drive unit 105 is a circuit that drives a tube bulb in the radiation source 2. The drive unit 105 is connected to the radiation source 2 with a cable.

The radio communication unit 106 is provided with an antenna 108 and wirelessly communicates with external units/devices, such as the FPD cassette 3.

The crystal oscillator 107 oscillates by a piezoelectric effect. The number of oscillations of the crystal oscillator 107 is sent to the CPU of the control unit 101. The control unit 101 counts time on the basis of the number of oscillations sent from the crystal oscillator 107.

The radiation source 2 can execute pulse irradiation. The radiation source 2 emits radiation (X-rays) to the subject H under the control of the radiation control device 1.

The FPD cassette 3 is a portable radiographic capturing apparatus capable of video capturing. The FPD cassette 3 is an indirect radiographic capturing apparatus including a scintillator that converts incident radiation into light having other wavelengths, such as visible light, and acquiring image data with the radiation detecting elements. Alternatively, the FPD cassette 3 may be a direct radiographic capturing apparatus that directly detects incident radiation with the radiation detecting elements without a scintillator.

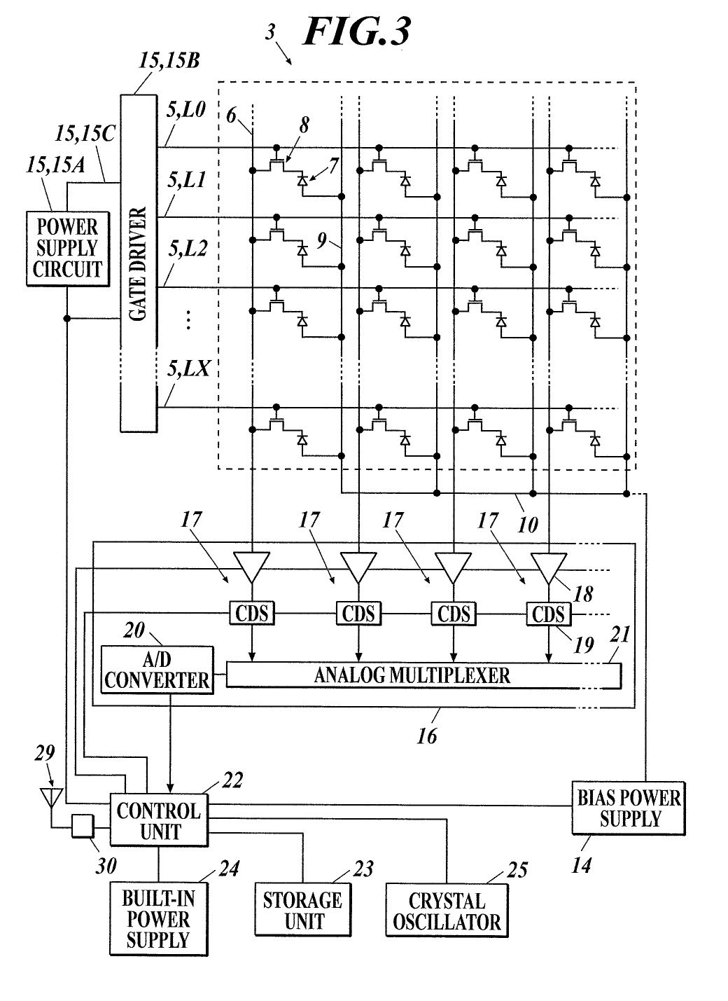

FIG. 3 is a block diagram illustrating an equivalent circuit of the FPD cassette 3. With reference to FIG. 3, the FPD cassette 3 includes a two-dimensional array (matrix) of radiation detecting elements 7 disposed on a sensor substrate (not shown) (a detecting unit). The radiation detecting elements 7 accumulate charges in proportion to the incident radiation. The radiation detecting elements 7 are connected to corresponding bias lines 9 that are connected to a connection 10. The connection 10 is connected to a bias power supply 14. An inverse bias voltage is applied to the radiation detecting elements 7 from the bias power supply 14 through the bias lines 9.

The radiation detecting elements 7 are connected to respective thin film transistors (TFTs) 8 as switching elements. The TFTs 8 are connected to corresponding signal lines 6. In a scan driving unit 15, a power supply circuit 15A supplies ON and OFF voltages to a gate driver 15B through a line 15C. The gate driver 15B switches the ON and OFF voltages and the switched voltage is applied to scanning lines 5(L0) to 5(Lx). The TFTs 8 are turned on in response to application of the ON voltage through the scanning lines 5 and cause the charges accumulated in the radiation detecting elements 7 to be discharged to the corresponding signal lines 6. The TFTs 8 are turned off in response to application of the OFF voltage through the scanning lines 5 and disconnects the radiation detecting elements 7 from the corresponding signal lines 6 to accumulate the charges generated in the radiation detecting elements 7 in the radiation detecting elements 7. Each of the radiation detecting elements 7 and the TFT 8 connected thereto constitute a pixel.

Multiple reading circuits 17 are provided in a reading IC 16 and connected to the respective signal lines 6. During the reading of image data, charges from the radiation detecting elements 7 flow into the reading circuits 17 through the signal lines 6, and voltage values in proportion to the charges are output from amplifier circuits 18. Correlated double sampling circuits (“CDSs” in FIG. 3) 19 read the voltage values output from the amplifier circuits 18 as image data having analog values, and output the image data to the components downstream. The image data are sequentially sent to an A/D converter 20 through an analog multiplexer 21, converted into image data having digital values at the A/D converter 20, and then stored in a storage unit 23.

A control unit 22 includes a computer (not shown) provided with a central processing unit (CPU), a read only memory (ROM), a random access memory (RAM), and an input/output interface which are connected to a bus, and a field programmable gate array (FPGA). The control unit 22 may be composed of a dedicated control circuit. The control unit 22 is connected to the storage unit 23 composed of a static RAM (SRAM), a synchronous DRAM (SDRAM), or a NAND flash memory, and to a radio communication unit 30 that establishes wireless communication with external units/devices, such as the radiation control device 1, via the antenna 29. The wireless communication established between the radiation control device 1 and the FPD cassette 3 eliminates the need for a cable connection between the radiation control device 1 and the FPD cassette 3 during capturing with the mobile medical cart. The operator can operate the mobile medical cart with an increased convenience.

The control unit 22 is connected to a built-in power supply 24 that supplies electrical power to the respective functional units, such as the scan driving unit 15, the reading circuits 17, the storage unit 23, and the bias power supply 14. As described above, the control unit 22 controls the operations of the components, such as the scan driving unit 15 and the reading circuits 17, to accumulate charges in proportion to the dose of radiation in the radiation detecting elements 7, cause the charges accumulated in the radiation detecting elements 7 to be discharged to the signal lines 6, and reads the discharged charges at the reading circuits 17 in the form of image data.

The control unit 22 is also connected to a crystal oscillator 25. The crystal oscillator 25 oscillates by a piezoelectric effect. The number of oscillations of the crystal oscillator 25 is sent to the CPU of the control unit 22. The control unit 22 counts time on the basis of the number of oscillations sent from the crystal oscillator 25. The time counted at the radiation control device 1 is synchronized with the time counted at the FPD cassette 3.

Although an operator or radiology technician may carry the FPD cassette 3, the relatively heavy FPD cassette 3 is readily damaged if dropped. Thus; the FPD cassette 3 can be placed in a cassette pocket 11 of the radiation control device 1 as the mobile medical cart.

### (Operation of Radiographic Capturing System 100)

The operation of the radiographic capturing system 100 will now be described.

The radiographic capturing system 100 according to this embodiment carries out calibration during installation (when initially introduced to a facility), for example. Calibration is the process of accumulating charges in proportion to the radiation emitted (preliminary for adjustment) from the radiation source 2 in at least some of the radiation detecting elements 7 and reading the accumulated charges, to obtain waveform information on the radiation emitted from the radiation source 2 at the FPD cassette 3, and adjusting the control conditions for generation of multiple frame images of a subject at the FPD cassette 3 on the basis of the obtained waveform information. The waveform information on the radiation (radiation output waveform information) identifies the waveform of the radiation, and corresponds to a charge amount proportional to an integrated value of the dose of radiation emitted from the radiation source 2 during the time from completion of reading by the radiation detecting elements 7 to the start of the next reading, for example.

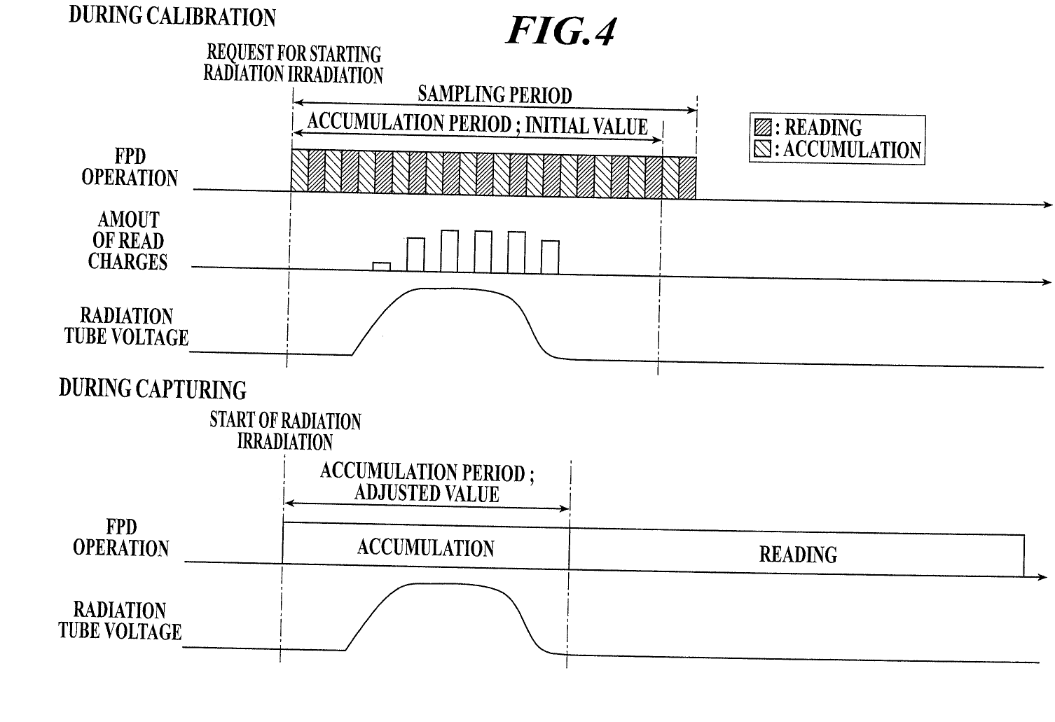

The upper portion of FIG. 4 illustrates the operation of the FPD cassette 3, the amount of read charges, and the radiation tube voltage, during calibration. The radiation source 2 emits radiation under the predetermined radiation irradiation conditions during calibration, as illustrated in the upper portion of FIG. 4. The FPD cassette 3 quickly alternates between accumulation and reading of charges in some of the two-dimensionally arrayed radiation detecting elements 7 (a single line of radiation detecting elements 7 in this case) during a predetermined calibration period, to obtain the waveform information of the radiation emitted from the radiation source 2 during the calibration period. The control conditions for generation of frame images of the subject are adjusted on the basis of the obtained waveform information.

The calibration of the radiographic capturing system 100 will now be described in detail.

First, the operator operates the operation unit 102 to instruct the radiation control device 1 to carry out calibration.

The operator then presses the first-stage switch of the exposure switch 102A. In response to the operation of the first-stage switch of the exposure switch 102A, the control unit 101 of the radiation control device 1 activates the radiation source 2 and instructs the radio communication unit 106 to send an activation signal and a calibration instruction signal to the FPD cassette 3 via the antenna 108. Upon reception of the activation signal and the calibration instruction signal by the radio communication unit 30, the control unit 22 of the FPD cassette 3 carries out a resetting process of the radiation detecting elements 7 by instructing the gate driver 15B of the scan driving unit 15 (see FIG. 3) to apply an ON voltage to a predetermined scanning line 5 to be used for the calibration so that the charges remaining in the radiation detecting elements 7 connected to the predetermined line are discharged to the signal lines 6, to remove the charges from radiation detecting elements 7. Upon completion of the resetting process, the control unit 22 instructs the gate driver 15B to apply an OFF voltage to the predetermined scanning line 5 to shift to a charge accumulation status. At the same time, the control unit 22 instructs the radio communication unit 30 to send an interlock release signal to the radiation control device 1.

When a second-stage switch of the exposure switch 102A is pressed, the control unit 101 of the radiation control device 1 determines whether the radio communication unit 106 has received the interlock release signal from the FPD cassette 3. If the control unit 101 determines that the interlock release signal has not been received, the control unit 101 waits for the reception of the interlock release signal. Upon reception of the interlock release signal, the control unit 101 instructs the radio communication unit 106 to send the radiation irradiation start time, which is to be used for adjustment, to the FPD cassette 3. When the radiation irradiation start time comes, the control unit 101 causes the drive unit 105 to instruct the radiation source 2 to emit radiation under the predetermined radiation irradiation conditions.

When the radiation irradiation start time notified by the radiation control device 1 comes in the FPD cassette 3, the control unit 22 performs a reading process to apply an ON voltage from the gate driver 15B to the predetermined scanning line 5 at a predetermined time interval to read the charges accumulated in the predetermined line. With reference to the upper portion of FIG. 4, the control unit 22 quickly alternates between accumulation and reading of the predetermined line during a predetermined sampling period. It is preferred that the sampling period be longer than a predetermined accumulation period (initial value). After the sampling period, the control unit 22 obtains the radiation output waveform information of the radiation source 2 on the basis of temporal variation in the amount of charges read at the predetermined time interval. The accumulation period is then adjusted on the basis of the obtained waveform information.

For example, the control unit 22 specifies the radiation irradiation period (from the radiation irradiation start time until the time radiation becomes undetectable) of the radiation source 2 on the basis of the radiation output waveform information of the radiation source 2. If the accumulation period (initial value) is longer than the specified radiation irradiation period as illustrated in the upper portion of FIG. 4, the accumulation period is shortened such that the accumulation period becomes approximately same as the radiation irradiation period as illustrated in the lower portion of FIG. 4. This shortens the entire image capturing period. This also improves the maximum frame rate. If the accumulation period (initial value) is shorter than the specified radiation irradiation period, the control unit 22 extends the accumulation period such that the accumulation period becomes approximately same as the radiation irradiation period. This avoids radiation irradiation during the reading period and thus prevents degradation of the next frame image due to the excess radiation irradiation.

The control unit 22 sets the adjusted accumulation period as the accumulation period for capturing, and stores the set accumulation period in the storage unit 23.

The capturing process of the radiographic capturing system 100 will now be described in detail.

The operator prepares for capturing by adjusting the radiation irradiation conditions and positioning the subject H, the radiation source 2, and the FPD cassette 3.

After the preparation for capturing, the operator presses the first-stage switch of the exposure switch 102A. In response to pressing of the first-stage switch of the exposure switch 102A, the control unit 101 of the radiation control device 1 activates the radiation source 2 and instructs the radio communication unit 106 to send an activation signal to the FPD cassette 3 via the antenna 108. Upon reception of the activation signal by the radio communication unit 30, the control unit 22 of the FPD cassette 3 carries out a resetting process of the radiation detecting elements 7 by instructing the gate driver 15B of the scan driving unit 15 (see FIG. 3) to sequentially apply an ON voltage to the scanning lines 5(L0) to 5(Lx) so that the charges remaining in the radiation detecting elements 7 are discharged to the signal lines 6, to remove the charges from the radiation detecting elements 7. Upon completion of the resetting process, the control unit 22 instructs the gate driver 15B to apply an OFF voltage to the scanning lines 5(L0) to 5(Lx) to shift to the charge accumulation status. At the same time, the control unit 22 instructs the radio communication unit 30 to send an interlock release signal to the radiation control device 1.

When the second-stage switch of the exposure switch 102A is pressed, the control unit 101 of the radiation control device 1 determines whether the radio communication unit 106 has received the interlock release signal from the FPD cassette 3. If the control unit 101 determines that the interlock release signal has not been received, the control unit 101 waits for the reception of the interlock release signal. Upon reception of the interlock release signal, the control unit 101 instructs the radio communication unit 106 to send the radiation irradiation start time for each frame image to the FPD cassette 3. When the radiation irradiation start time comes, the control unit 101 causes the drive unit 105 to instruct the radiation source 2 to execute the radiation irradiation (pulse irradiation).

When the radio communication unit 30 of FPD cassette 3 receives the radiation irradiation start time from the radiation control device 1, the control unit 22 waits until the radiation irradiation start time comes. When the radiation irradiation start time comes, the control unit 22 instructs the radiation detecting elements 7 to accumulate charges during the accumulation period stored in the storage unit 23. The control unit 22 then instructs the gate driver 15B to sequentially apply an ON voltage to the scanning lines 5(L0) to 5(Lx), as described above, to perform the reading process to read the image data of the frame images.

The control unit 22 repeats the accumulation process and the reading process for all frame images, to generate multiple frame images constituting the moving image of the subject H.

The control unit 22 of the FPD cassette 3 adjusts the accumulation period on the basis of the radiation output waveform information of the radiation source 2 obtained through the calibration. In the calibration, if the accumulation period (initial value) is longer than the radiation irradiation period as illustrated in the upper portion of FIG. 4, the accumulation period is shortened such that the accumulation period becomes approximately same as the radiation irradiation period, and is defined as the accumulation time for capturing. This shortens the entire capturing period. This also improves the maximum frame rate. In the calibration, if the accumulation period (initial value) is shorter than the radiation irradiation period in the calibration, the accumulation period is extended as to be approximately same as the radiation irradiation period, and is defined as the accumulation period for capturing. This prevents degradation of the images due to excess radiation irradiation outside of the accumulation period.

### Second Embodiment

The second embodiment will now be described.

In the first embodiment, calibration is preliminarily carried out during installation of the radiographic capturing system 100 or at other timings to obtain the radiation output waveform information of the radiation source 2 and adjust the accumulation period on the basis of the obtained waveform information. In the second embodiment, calibration is preliminarily carried out during installation of the radiographic capturing system 100 or at other timings to obtain the radiation output waveform information of the radiation source 2, adjust image correction factors on the basis of the obtained waveform information, and correct the frame images with the image correction factors during capturing.

The configuration of the radiographic capturing system according to the second embodiment is identical to that of the radiographic capturing system 100 according to the first embodiment. Thus, description thereof is omitted. The calibration according to the second embodiment is identical to that of the first embodiment up to obtaining the radiation output waveform information of the radiation source 2 by the control unit 22 of the FPD cassette 3. Thus, the descriptions thereof are omitted.

In the calibration, upon acquisition of the radiation output waveform information of the radiation source 2, the control unit 22 of the FPD cassette 3 adjusts the image correction factors on the basis of the obtained radiation output waveform information.

In detail, in the calibration, the control unit 22 determines the charge amount detected through reading outside of the accumulation period, and the time of the detection of the charge amount, on the basis of the radiation output waveform information. The control unit 22 then determines the lines irradiated with radiation during the charge reading for capturing, and the charge amount in proportion to the emitted radiation, on the basis of the determined charge amount and time. The control unit 22 then determines the image correction factors α(γ) for the respective lines on the basis of the determined lines and charge amount, and stores the factors α(γ) in the storage unit 23.

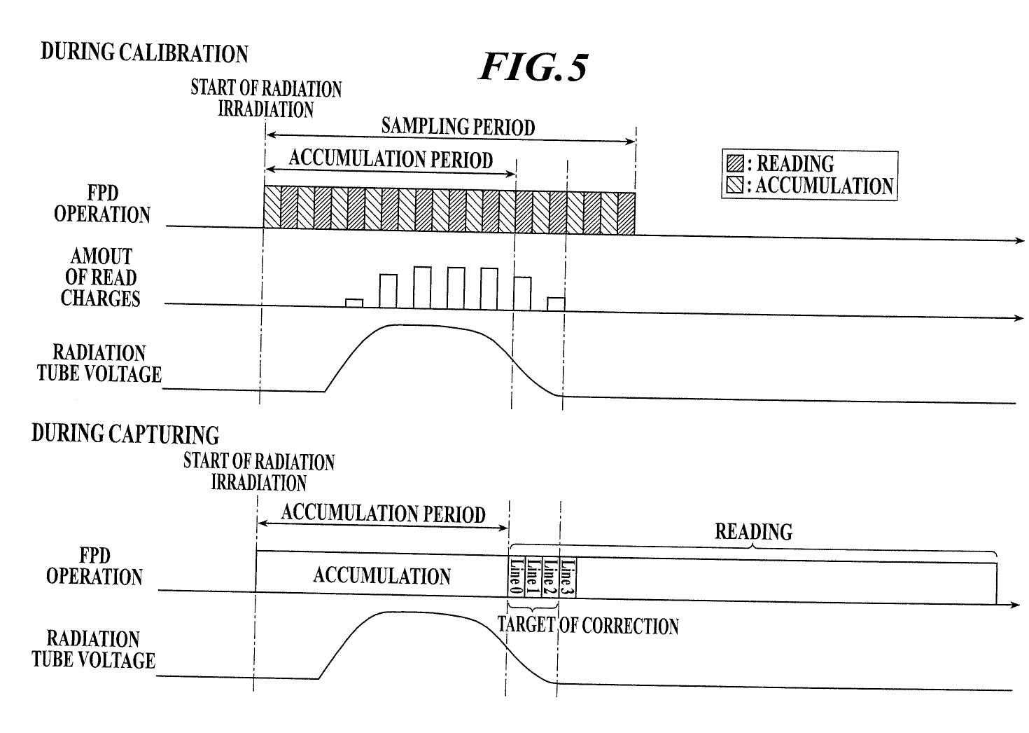

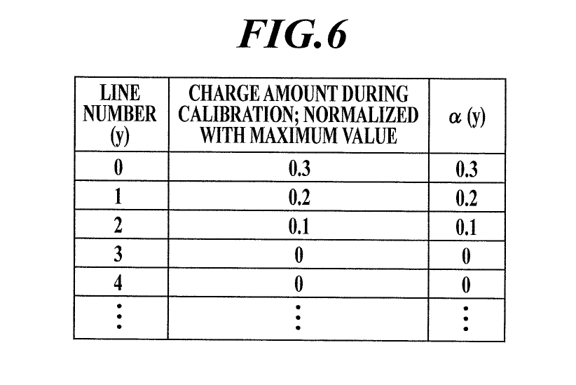

In the example illustrated in FIG. 5, the lines L0 to L2 are determined to be irradiated with radiation during the charge reading for capturing on the basis of the radiation output waveform information obtained through calibration. Then, the image correction factors α(γ) of the lines L0 to Lx are determined on the basis of the charge amount which has been detected outside of the accumulation period in the calibration. The line number (γ), the charge amount in the line during calibration, and the factor α(γ) are correlated with one another and stored in the storage unit 23, as illustrated in FIG. 6.

The image capturing process according to the second embodiment will now be described in detail.

The operator prepares for capturing by adjusting the radiation irradiation conditions and positioning the subject H, the radiation source 2, and the FPD cassette 3.

After completion of the preparation, the operator presses the first-stage switch of the exposure switch 102A. In response to pressing of the first-stage switch of the exposure switch 102A, the control unit 101 of the radiation control device 1 activates the radiation source 2 and instructs the radio communication unit 106 to send an activation signal to the FPD cassette 3 via the antenna 108. Upon reception of the activation signal by the radio communication unit 30, the control unit 22 of the FPD cassette 3 carries out a resetting process of the radiation detecting elements 7 by instructing the gate driver 15B of the scan driving unit 15 (see FIG. 3) to sequentially apply an ON voltage to the scanning lines 5(L0) to 5(Lx) so that the charges remaining in the radiation detecting elements 7 are discharged to the signal lines 6, to remove the charges from radiation detecting elements 7. Upon completion of the resetting process, the control unit 22 instructs the gate driver 15B to apply an OFF voltage to the scanning lines 5(L0) to 5(Lx) to shift to the charge accumulation status. At the same time, the control unit 22 instructs the radio communication unit 30 to send an interlock release signal to the radiation control device 1.

When the second-stage switch of the exposure switch 102A is pressed, the control unit 101 of the radiation control device 1 determines whether the radio communication unit 106 has received an interlock release signal from the FPD cassette 3. If the control unit 101 determines that the interlock release signal has not been received, the control unit 101 waits for the reception of the interlock release signal. Upon reception of the interlock release signal, the control unit 101 instructs the radio communication unit 106 to send the radiation irradiation start time for each frame image to the FPD cassette 3. When the radiation irradiation start time comes, the control unit 101 causes the drive unit 105 to instruct the radiation source 2 to execute the radiation irradiation (pulse irradiation).

In the FPD cassette 3, when the radio communication unit 30 receives the radiation irradiation start time from the radiation control device 1, the control unit 22 waits until the radiation irradiation start time comes. When the radiation irradiation start time comes, the control unit 22 instructs the radiation detecting elements 7 to accumulate charges during the accumulation period, and then instructs the gate driver 15B to sequentially apply an ON voltage to the scanning lines 5(L0) to 5(Lx), as described above, to perform the reading process to read the image data of the frame images. For the second and subsequent frame images, the image data of the immediately previous frame image is multiplied by the image correction factor α(γ) determined through calibration, and subtracted from the image data of the current frame image read from the lines, as defined in Expression 1.

dₒᵤₜ(x,y,t)=dᵢₙ(x,y,t)−α(y)×dᵢₙ(x,y,t−1) (Expression 1)

where dₒᵤₜ(x,y,t) is the corrected image data at coordinates (x,y) at time t, and dᵢₙ(x,y,t) is the read image data at the coordinates (x,y) at time t.

The image correction factors α(γ) are zero for the lines not irradiated with radiation during the charge reading for capturing. Thus, calibration (subtraction) is substantially omitted for these lines.

When the radiation irradiation period is longer than the accumulation period and radiation is emitted during the reading period, the process described above can prevent image degradation due to radiation emitted during the reading period.

As described above, the control unit 22 of the FPD cassette 3 of the radiographic capturing system 100 preliminarily carries out calibration, for adjustment, involving accumulation of charges in proportion to the radiation emitted from the radiation source 2 in at least some of the radiation detecting elements 7 and reading of the accumulated charges, to obtain waveform information on the radiation emitted from the radiation source 2 and adjust the control conditions for generation of multiple frame images of a subject on the basis of the obtained waveform information.

The control unit 22 determines the radiation irradiation period of the radiation source 2 on the basis of the waveform information obtained through calibration, and if the charge accumulation period is longer than the radiation irradiation period, the control unit 22 shortens the charge accumulation period such that the charge accumulation period becomes approximately same as the radiation irradiation period, for example. Thus, drawbacks can be prevented, such as ineffectively long capturing time, due to the difference between the radiation irradiation period and the charge accumulation period caused by the radiation output characteristics of the radiation source 2.

The control unit 22 determines the radiation irradiation period of the radiation source 2 on the basis of the waveform information obtained through calibration, and if the charge accumulation period is shorter than the radiation irradiation period, the control unit 22 extends the charge accumulation period such that the charge accumulation period becomes approximately same as the radiation irradiation period, for example. Thus, drawbacks can be prevented, such as degradation of images due to radiation irradiation during the charge reading due to the difference between the radiation irradiation period and the charge accumulation period caused by the radiation output characteristics of the radiation source 2.

The control unit 22 determines, among multiple lines of the radiation detecting elements 7, the line irradiated with radiation during the charge reading for generation of the multiple frame images of the subject, and adjusts the image correction factor for the determined line, on the basis of the obtained waveform information. With respect to the image data of the second and subsequent frame images among multiple frame images of the subject, the control unit 22 multiplies the image correction factor by the image data corresponding to the determined line of the immediately previous frame image, and subtracts the resulting image data from the image data corresponding to the current frame. Thus, drawbacks can be prevented, such as degradation of images due to radiation irradiation during the charge reading due to the difference between the radiation irradiation period and the charge accumulation period caused by the radiation output characteristics of the radiation source 2.

The embodiments of the present invention should not be limited to those described above.

In the embodiments described above, accumulation and reading of charges of a single line are repeated during calibration. Alternatively, charges in multiple lines or multiple pixels may be accumulated and read.

The detailed configuration and operation of the components of the radiographic capturing system may be appropriately modified without departing from the scope of the present invention.

What is claimed is:

1. A portable radiographic capturing apparatus comprising:
a detecting unit provided with a two-dimensional array of radiation detecting elements each of which accumulates one or more charges in proportion to a dose of radiation; and
a control unit which controls accumulation of the charges by the radiation detecting elements and reading of the accumulated charges from the radiation detecting elements, the charges being in proportion to the dose of radiation emitted as pulsed radiation from a radiation source and transmitted through a subject, to generate a plurality of frame images of the subject, wherein
the control unit obtains waveform information on the radiation emitted from the radiation source by causing at least some of the radiation detecting elements to accumulate the charges in proportion to the radiation preliminarily emitted from the radiation source for adjustment and by reading the accumulated charges, and adjusts a control condition for generation of the frame images of the subject on the basis of the obtained waveform information.

2. The radiographic capturing apparatus of claim 1, wherein the control unit adjusts a charge accumulation period of the radiation detecting elements on the basis of the obtained waveform information.

3. The radiographic capturing apparatus of claim 2, wherein the control unit determines a radiation irradiation period of the radiation source on the basis of the obtained waveform information, and shortens the charge accumulation period such that the charge accumulation period becomes approximately same as the radiation irradiation period if the charge accumulation period is longer than the radiation irradiation period.

4. The radiographic capturing apparatus of claim 2, wherein the control unit determines a radiation irradiation period of the radiation source on the basis of the obtained waveform information, and extends the charge accumulation period such that the charge accumulation period becomes approximately same as the radiation irradiation period if the charge accumulation period is shorter than the radiation irradiation period.

5. The radiographic capturing apparatus of claim 1, wherein
the control unit determines, among a plurality of lines of the radiation detecting elements, a line irradiated with the radiation during charge reading for generation of the frame images of the subject and adjusts an image correction factor for the determined line on the basis of the obtained waveform information, and with respect to image data of a second and subsequent frame images among the frame images of the subject, the control unit multiplies the image correction factor by image data corresponding to the determined line of an immediately previous frame image, and subtracts the resulting image data from image data of a current frame.

6. A radiographic capturing system comprising:
a radiation source capable of emitting pulsed radiation; and
the radiographic capturing apparatus of claim 1.
